(19) United States
(12) Patent Application Publication
(10) Pub. No.: US 2019/0262407 A1
(43) Pub. Date: Aug. 29, 2019

(54) PROBIOTIC COMPOSITIONS AND METHODS OF USE THEREOF

(71) Applicant: LIFEBRIDGE HEALTH, INC., Baltimore, MD (US)

(72) Inventor: Sudhir Kumar Dutta, Baltimore, MD (US)

(21) Appl. No.: 16/282,538

(22) Filed: Feb. 22, 2019

Related U.S. Application Data
(60) Provisional application No. 62/634,484, filed on Feb. 23, 2018.

(51) Int. Cl. A61K 35/742; A61P 1/14; A61P 1/12; A61K 35/745; A61K 35/747; A61K 9/00; A61K 9/48

(52) U.S. Cl. CPC A61K 35/742; A61P 1/14; A61P 1/12; A61K 35/745; A61K 9/0031; A61K 9/0056; A61K 9/4825; A61K 9/4808; A61K 35/747

(57) ABSTRACT

Various probiotic therapies including butyrogenic bacteria are provided for the treatment of gastrointestinal diseases caused by microbial dysbiosis. Butyrogenic bacteria produce the short chain fatty acid, butyric acid. Gastrointestinal disorders that are associated with microbial dysbiosis include but are not limited to, inflammatory bowel disease, such as Crohn's disease and ulcerative colitis, irritable bowel syndrome, recurrent Clostridium difficile infection, and antibiotic-associated diarrhea.

### CROSS REFERENCE TO RELATED APPLICATIONS

This application claims benefit of and priority to U.S. Provisional Patent Application No. 62/634,484 filed on Feb. 23, 2018, which is incorporated herein by reference in its entirety.

### FIELD OF THE INVENTION

This invention generally relates to probiotic compositions and methods of their use thereof to treat dysbiosis in the human microbiome associated with gastrointestinal disorders.

### BACKGROUND OF THE INVENTION

The human microbiome consists of 10-100 trillion symbiotic microbial cells harbored in the human body, predominantly in the human intestinal tract. The bacteria, archae, and eukarya colonizing the gastrointestinal tract are collectively called the gut microbiome. Over the past decade, the microbiome has emerged as a critical component in the regulation of host health. Microbes perform many processes that the human body cannot handle itself. For example, microbes digest food to generate nutrients for host cells, synthesize vitamins, metabolize drugs, detoxify carcinogens, stimulate renewal of cells in the gut lining and activate and support the immune system. Emerging evidence also suggests that the microbiota play a role in the pathogenesis of many diseases and disorders.

Dysbiosis in the gastrointestinal (GI) microbiome is associated with various GI disorders such as C. difficile colitis, inflammatory bowel diseases (IBD) such as ulcerative colitis (UC) and Crohn's disease, irritable bowel syndrome (IBS), Celiac disease, and general antibiotic-associated microbial dysbiosis. Therefore, manipulation of the microbiome may be crucial for the treatment of these GI disorders. There have been some successes in the manipulation of the microbiome to counteract dysbiosis. Diet exerts major effects on the gut microbiota and is one of the main drivers in shaping the gut microbiome over time. The consumption of food or dietary supplements containing prebiotics or dietary fiber provides fuel for the bacteria residing in the gut and has been shown to reduce symptoms associated with various GI diseases and disorders. Fecal microbiota transplantation has emerged as a potentially beneficial method for manipulating the gut microbiome. In this procedure, stool from a healthy donor is administered to an unhealthy subject. Changes in the microbiome of recipients have been observed, and are likely due to the presence of members of the healthy microbiota in the transplanted stool from healthy patients colonizing the unhealthy gut and re-establishing normal GI function and metabolic niches. While this therapy has been successful in the clinic, it has not been widely accepted because of concerns over infection transmission, lack of aesthetic appeal, and lack of knowledge of long-term effects within the recipient microbiome.

Another more widely accepted method for restoring the gut microbiome is the administration of probiotics. Probiotics are isolated colonies of live microorganisms that confer benefits to the host. These microorganisms are usually made up of commensal bacteria encapsulated for daily use. While there is growing evidence to support the use of probiotics for gastrointestinal health, there are limitations in the field of probiotics. Currently many probiotics are composed of the same few commensal bacteria of the families Lactobacillus and Bifidobacterium. Additionally, the number of CFUs and specific delivery system to achieve efficient colonization are still relatively unknown. Therefore, there is a need for the discovery of new probiotic compositions to combat gastrointestinal dysbiosis.

Several studies have shown that alterations in the human microbiome are characterized by a reduction in the diversity of bacterial families and disequilibrium in various groups of microbiota. Anaerobes in the colon ferment polysaccharides, which results in the production of short chain fatty acids (SCFA) (Hooper, et al., Annual Review of Nutrition, 22:283-307 (2002); Pryde, et al., FEMS Microbiology Letters, 217:133-139 (2002)). The three main SCFAs are proprionate, acetate, and butyrate. SCFAs are the major energy source in the intestines, contributing 60-70% of the energy derived from carbohydrates (Bergman, et al., Physiol Rev, 70:567-590 (1990)). SCFAs also contribute to host health through epithelial proliferation and differentiation. Butyrate in particular is considered to have an important role in regulation of digestive health (Pryde, et al., FEMS Microbiology Letters, 217:133-139 (2002)).

Depletion of specific bacterial families such as Lachnospiracea and other butyric-acid producing bacteria are common in GI disorders. The depletion of butyric-acid producing bacteria is implicated in the development of colonic inflammation that is characteristic of C. difficile colitis, inflammatory bowel disease (IBD), irritable bowel disorder (IBS), and general antibiotic-associated microbial dysbiosis. The importance of butyrate in the GI tract has been known for some time, but the connection between the gut microbiome and this crucial SCFA was not made until recently. Colonic epithelial cells use butyrate as one of their main sources of energy (Roediger, 1980). In addition to its role in fueling colonic cells, butyrate is also key in regulating cellular proliferation and differentiation (Wong et al., 2006). It has also been shown to produce anti-inflammatory effects by inhibiting the activation of transcription factor NF-KB, which leads to a reduction in proinflammatory cytokines (Luhrs et al., 2001; Segain et al., 2000). There is evidence that butyrate supplements can be effective in ameliorating the inflammation caused by various GI diseases and disorders, including C. difficile colitis, Crohn's disease, and IBS. Several studies, including a clinical trial of Crohn's patients, have demonstrated that butyrate treatment can reduce markers of inflammation and improve disease symptoms (Segain, et al., (2000); Sabatino, et al, (2005)).

Therefore, it is an object of the invention to provide methods and compositions for treating dysbiosis in the human gut microbiome.

It is also an object of the invention to provide probiotic compositions for the treatment of dysbiosis.

### SUMMARY OF THE INVENTION

Probiotic compositions and methods of their use for treating gastrointestinal dysbiosis are provided. One embodiment provides a probiotic composition for the treatment of gastrointestinal dysbiosis including an effective amount viable, non-pathogenic human gut microbes, wherein at least one of the microbes produces butyric acid. In addition to the effective amount of at least one butyrogenic bacteria, the probiotic composition can also include at least one Bifidobacterium spp., at least one Lactobacillus spp., and S. boulardii. For example, the probiotic composition can contain 25% butyrogenic bacteria, 25% Bifidobacterium spp, 25% Lactobacillus spp., and 25% S. boulardii.

In one embodiment, the butyrogenic bacteria is selected from the group consisting of Clostridium butyricum, Eubacterium limosum, Eubacterium rectale, Faecalibacterium prausnitzii, Roseburia faecis, and Roseburia intestinalis.

One embodiment provides a probiotic composition having Clostridium butyricum, Faecalibacterium prausnitzii, Bifidobacterium spp., Lactobacillus spp., and S. boulardii. Another probiotic composition contains Eubacterium limosum, Eubacterium rectale, Clostridium butyricum, Faecalibacterium prausnitzii, Bifidobacterium spp., Lactobacillus spp., and S. boulardii. In another embodiment, the probiotic composition contains Roseburia faecis, Roseburia intestinalis, Clostridium butyricum, Faecalibacterium prausnitzii, Bifidobacterium spp., Lactobacillus spp., and S. boulardii. Yet another embodiment provides a probiotic composition including Clostridium butyricum, Faecalibacterium prausnitzii, Bifidobacterium spp., Lactobacillus spp., and S. boulardii.

One embodiment provides a probiotic composition that is formulated for oral administration. The composition can be formulated as a time controlled capsule, a pH controlled capsule, an enzyme controlled capsule, or a combination thereof. In another embodiment, the probiotic is formulated for rectal administration.

Another embodiment provides a method of treating gastrointestinal dysbiosis by administering to a subject in need thereof any of the disclosed probiotic compositions having an effective amount of viable, non-pathogenic human gut microbes, wherein at least one of the microbes produces butyric acid. The gastrointestinal dysbiosis can be the cause of gastrointestinal diseases in the subject in need thereof. In one embodiment, the subject in need thereof has recurrent C. difficile infection, inflammatory bowel disease such as Crohn's disease or ulcerative colitis, irritable bowel syndrome, or antibiotic-associated bacterial dysbiosis. The probiotic composition can be administered to the subject orally or rectally.

### DETAILED DESCRIPTION OF THE INVENTION

I. Definitions

As used herein, the term “probiotic composition” refers to a product containing at least one live probiotic bacterial strain.

As used herein, “probiotics” are live bacteria or yeast that when consumed confer a health benefit to the host. Probiotics are said to restore the balance of bacteria in the gut when it has become disrupted through long-term antibiotic use or gastrointestinal disease. Examples of probiotics include but are not limited to Bifidobacterium spp., Lactobacillus spp., Streptococcus thermophilia, Bacillus coagulans, Bacillus laterosporus, Pediococcus acidilactici, Saccharomyces boulardii.

As used herein, a “prebiotic” is a selectively fermented ingredient that allows specific changes, both in the composition and/or activity of the gastrointestinal microflora that confers benefits upon host well-being and health. Examples of prebiotics include but are not limited to inulin, arabinoxylan, xylose, soluble fiber dextran, soluble corn fiber, polydextrose, lactose, N-acetyl-lactosamine, glucose, galactose, fructose, rhamnose, mannose, uronic acids, 3′-fucosyllactose, 3′-sialylactose, 6′-sialyllactose, lacto-N-neotetraose, 2′-2′-fucosyllactose, trans-galactooligosaccharides, glucooligosaccharides, isomaltooligosaccharides, lactosucrose, polydextrose, soybean oligosaccharides, and arabinose, cellobiose, fructose, fucose, galactose, glucose, lactose, lactulose, maltose, mannose, ribose, sucrose, trehalose, xylobiose, xylooligosaccharide, D-xylose, and xylitol.

As used herein, the terms “gut flora”, “gastrointestinal flora”, “intestinal flora”, “gut microbiome”, “intestinal microbiome”, and “microbiome” are interchangeable and are intended to represent the normal, naturally occurring bacterial population present in the gastric and intestinal systems of healthy humans and animals. It is meant to reflect both the variety of bacterial species and the concentration of bacterial species found in a healthy human or animal.

As used herein, the terms “gut”, “intestine”, “intestinal tract”, and “colon” are used interchangeably and are intended to represent the intestinal system of humans.

As used herein, the term “dysbiosis” refers to a microbial imbalance on or within the body. More specifically as used herein, the microbial imbalance refers to one within the gut microbiome.

As used herein, the term “synbiotic” refers to a product that contains both a prebiotic and a probiotic.

As used herein, “C. difficile” refers to the bacterium Clostridium difficile. C. difficile is a spore-forming bacterium that is especially prevalent in soil. C. difficile causes debilitating and sometimes deadly colitis in humans, and is the leading cause of antibiotic-associated diarrhea. The primary symptom of C. difficile infection (CDI) is watery diarrhea, which also acts as the primary mode of transmission (Centers for Disease Control and Prevention, 2016). It is estimated that C. difficile caused approximately 453,000 infections and was associated with 29,000 deaths in the US in 2011, with highest incidence in individuals 65 years or older, whites, and females (Lessa, et al., N Engl J Med, 372:825-834 (2015)). The recommended treatments for CDI are vancomycin or fidaxomicin, but these antibiotics are not effective for every patient and recurrent infection is common in some patients (CDC, 2016).

As used herein, the abbreviation “RCDI” stands for recurrent C. difficile infection. RCDI includes patients with 2 or more incidences of CDI after discontinuation of antibiotic therapy. There are several antibiotics that have been used successfully in treatment of CDI including metronidazole, vancomycin and fidaxomicin. However, in RCDI patients there is only a partial resolution of symptoms with these antibiotics and recurrence of CDI after the discontinuation of these antibiotics. Interestingly, fecal microbiota transplantation (FMT) is a highly effective method of treatment of RCDI.

As used herein, “short chain fatty acids” (SCFA) refer to the group of fatty acids with less than six carbons that are produced by anaerobes of the human large intestine. Certain types of gut bacteria ferment indigestible polysaccharides, resulting in the production of three major SCFAs: acetate, propionate, and butyrate (Hooper, et al., Annual review of Nutrition, 22:283-307 (2002); Pryde et al., FEMS Microbiology Letters, 217:133-139 (2002)). SCFAs are a major source of energy not only for enterocytes but also for the entire body. In fact, it is estimated that 60-75% of the energy derived from ingested carbohydrates can be attributed to SCFA production (Bergman, Physiol Rev, 70:567-590 (1990)). In addition to their contribution to host metabolism, SCFAs also influence colonic health through regulation of epithelial proliferation and differentiation (den Besten et al., Journal of Lipid Research, 54:2325-2340 (2013); Hijova & Chmelarova, 2007; Kripke et al., Journal of Parenteral and Enteral Nutrition, 13:109-116 (1989); Mortensen & Clausen, Scandinavian Journal of Gastroenterology, 216:132-148 (1996); Vinolo et al., Nutrients, 3:858-876 (2011); Wong et al., Journal of Clinical Gastroenterology, 40:235-243 (2006)).

As used herein, “butyrate” is one of the SCFAs and is considered to have an important role in the regulation of digestive health (Pryde et al., Oman Medical Journal, 25:79-87 (2002)). The importance of butyrate in the GI tract has been known for some time, but the connection between the gut microbiome and this crucial SCFA was not made until recently. Colonic epithelial cells prefer butyrate as a food source. It is estimated that 70% to 90% of produced butyrate is metabolized by colonocytes (Wong et al., Journal of Clinical Gastroenterology, 40:235-243 (2006)), and overall the colonic epithelium obtains 60-70% of its required energy from butyrate (Roediger, Gut, 793-798 (1980)).

As used herein, “colonic butyrogenic bacteria” refer to intestinal bacteria that produce butyrate as a byproduct of fermentation. They are Gram-positive Firmicutes with high phylogenetic diversity. The most abundant groups of butyrate producers include Eubacterium rectale, Roseburia spp., and Faecalibacterium prausnitzii (Louis & Flint, FEMS Microbiology Letters, 294:1-8 (2009)).

As used herein, “Clostridium butyricum” is a butyrate-producer and known member of the healthy human gut microbiome. C. butyricum has been shown to regulate gut homeostasis and an anti-inflammatory response by inducing inflammatory cytokine IL-10 macrophages through Toll-like receptor 2 or myeloid differentiation primary response gene 88 pathway (Kanai et al., J Gastroenterol, 50:928-939 (2015); Hayashi et al., Cell Host and Microbe, 13:711-722 (2013)).

As used herein, “Eubacterium limosum” is a member of the healthy human gut microbiome, and the administration of E. limosum amongst other bacteria during C. difficile infection has been shown to combat the C. difficile and improve colitis symptoms (Petrof et al., Microbiome, 1:1-12 (2013); Martz et al., Journal of Gastroenterology, 52:452-465 (2016)).

As used herein, “Faecalibacterium prausnitzii” is one of the most important groups of butyrate-producers in the healthy human gut (Canani et al., World Journal Gastroenterology, 17:1519-1528 (2011)). It is also considered to be one of the most abundant bacteria in the healthy human gut microbiome, with an estimated representation of 5% out of the overall pool of commensal microbiota (Miguel et al., Opinion in Mocrobiology, 16:255-261 (2013)). The anti-inflammatory effects of F. prausnitzii have been demonstrated in vitro and in vivo in the context of Crohn's disease (CD) and other inflammatory bowel diseases (Martin et al., BMC Microbiology, 15:1-12 (2015); Sokol et al., PNAS, 105:16731-16736 (2008); Rossi et al., Nature Scientific Reports, 6:1-12 (2016)).

As used herein, “Roseburia faecis” and “Roseburia intestinalis” are butyrate producing bacteria. The administration of R. faecis and R. intestinalis amongst other bacteria during C. difficile infection has been shown to improve colitis symptoms and combat the infection itself (Petrof et al., Microbiome, 1:1-12 (2013); Martz et al., Journal of Gastroenterology, 52:452-465 (2016).

As used herein, the abbreviation “CFU” stand for colony forming unit and refers to the amount of bacteria in a probiotic that are viable and capable of dividing and forming colonies.

II. Probiotic Compositions

Probiotic compositions and methods of their use in the treatment of various gastrointestinal diseases and disorders are provided. One embodiment provides probiotic compositions containing butyrogenic bacteria that treat dysbiosis in the human GI microbiome, and are useful in the treatment of various GI disorders. Representative GI disorders include but are not limited to C. difficile colitis, IBS, IBD, and antibiotic-associated microbial dysbiosis.

A. Commensal Microbiota

The human intestine is one of the most microbially diverse organs in the human body. The gut is host to up to 1000 different species of bacteria. Gut microbes perform many processes that the human body cannot handle itself, for example microbes digest food to generate nutrients for host cells, synthesize vitamins, metabolize drugs, detoxify carcinogens, stimulate renewal of cells in the gut lining and activate and support the immune system.

In one embodiment, the disclosed probiotic compositions include one or more commensal microbes. The most abundant phyla in the gut microbiome are Firmicutes, Acinobacteria, Proteobacteria, and Bacteriodes. Additionally most belong to the genera Bacteroides, Clostridium, Faecalibacterium, Eubacterium, Ruminococcus, Peptococcus, Peptostreptococcus, and Bifidobacterium. In one embodiment, the disclosed probiotic compositions include commensal bacteria Bifidobacterium spp. and Lactobacillus spp.

SCFA are an important metabolite of commensal gut microbes. The disclosed probiotic compositions include one or more butyrogenic species of bacteria. In one embodiment, the butyrogenic bacteria can be any one of, Clostridium butyricum, Eubacterium limosum, Eubacterium rectale, Faecalibacterium prausnitzii, Roseburia faecis, and Roseburia intestinalis.

B. Designer Probiotic Compositions

One embodiment provides a probiotic composition that contains butyrogenic bacteria and commensal gut bacteria. The probiotic composition can contain 5-100% butyrogenic bacteria, and commonly used microorganisms such as Bifidobacterium spp., Lactobacillus spp., and S. boulardii included in ratios of 1:1:1. This will be adjusted according to the percentage of butyrogenic bacteria included.

In one embodiment, the probiotic composition is used to treat RCDI. The disclosed composition for RCDI contains the bacterial strains Clostridium butyricum, Faecalibacterium prausnitzii, Bifidobacterium spp., Lactobacillus spp., and S. boulardii. The butyrogenic bacteria (C. butyricum and F. prausnitzii) will comprise 5-100% of the total composition. The remaining Bifidobacterium spp., Lactobacillus spp., and S. boulardii will be included in a 1:1:1 ratio depending upon the percentage of butyrogenic bacteria included in the final composition.

In another embodiment, the probiotic composition is used to treat IBD. The disclosed composition for IBD contains the bacterial strains Eubacterium limosum, Eubacterium rectale, Clostridium butyricum, Faecalibacterium prausnitzii, Bifidobacterium spp., Lactobacillus spp., and S. boulardii. The butyrogenic bacteria (E. limosum, E. rectale, C. butyricum, and F. prausnitzii) will comprise 5-100% of the total composition. The remaining Bifidobacterium spp., Lactobacillus spp., and S. boulardii will be included in a 1:1:1 ratio depending upon the percentage of butyrogenic bacteria included in the final composition.

One embodiment provides a probiotic composition that is used to treat IBS. The probiotic composition for IBS includes the bacterial strains Roseburia faecis, Roseburia intestinalis, Clostridium butyricum, Faecalibacterium prausnitzii, Bifidobacterium spp., Lactobacillus spp., and S. boulardii. The butyrogenic bacteria (R. faecis, R. intestinalis, C. butyricum, and F. prausnitzii) will make up 5-100% of the total composition. The remaining Bifidobacterium spp., Lactobacillus spp., and S. boulardii will be included in a 1:1:1 ratio depending upon the percentage of butyrogenic bacteria included in the final composition.

In one embodiment the probiotic composition is used to treat antibiotic associated bacterial dysbiosis. The probiotic composition for antibiotic associated bacterial dysbiosis include the bacterial strains Clostridium butyricum, Faecalibacterium prausnitzii, Bifidobacterium spp., Lactobacillus spp., and S. boulardii. The butyrogenic bacteria (C. butyricum and F. prausnitzii) will make up 5-100% of the total composition. The remaining Bifidobacterium spp., Lactobacillus spp., and S. boulardii will be included in a 1:1:1 ratio depending upon the percentage of butyrogenic bacteria included in the final composition.

The total bacterial volume in the probiotic composition will be between 10⁸ and 10¹² CFU per dose. In one embodiment, the probiotic composition includes 10³-10¹² CFU each of a butyrogenic bacterial species, Bifidobacterium spp, Lactobacillus spp., and S. boulardii.

C. Prebiotic Compositions

In some embodiments, the probiotic composition includes a prebiotic. Prebiotics are selectively fermented ingredients that stimulate the growth and/or activity of one or a limited number of bacteria in the gastrointestinal flora that confers benefits upon host well-being and health. Examples of prebiotics include but are not limited to inulin, arabinoxylan, xylose, soluble fiber dextran, soluble corn fiber, polydextrose, lactose, N-acetyl-lactosamine, glucose, galactose, fructose, rhamnose, mannose, uronic acids, 3′-fucosyllactose, 3′-sialylactose, 6′-sialyllactose, lacto-N-neotetraose, 2′-2′-fucosyllactose, trans-galactooligosaccharides, glucooligosaccharides, isomaltooligosaccharides, lactosucrose, polydextrose, pectin, soybean oligosaccharides, and arabinose, cellobiose, fructose, fucose, galactose, glucose, lactose, lactulose, maltose, mannose, ribose, sucrose, trehalose, xylobiose, xylooligosaccharide, D-xylose, and xylitol. Probiotic compositions can include a daily dose of prebiotics in the range 5 g-20 g.

In one embodiment, the probiotic composition includes dietary fiber. Dietary fiber is the indigestible portion of food produced by plants. It has a wide-range of health benefits including lower risk of heart disease and maintenance of gut health. Dietary fiber can be included in the probiotic composition disclosed herein at a daily range of 2.5 g-5 g.

D. Pharmaceutical Compositions

Pharmaceutical compositions including the disclosed probiotic compositions are provided. Pharmaceutical compositions containing the probiotic compositions can be formulated for administration by enteral routes of administration such as oral or rectal routes.

In some in vivo approaches, the compositions disclosed herein are administered to a subject in a therapeutically effective amount. As used herein the term “effective amount” or “therapeutically effective amount” means a dosage sufficient to treat, inhibit, or alleviate one or more symptoms of the disorder being treated or to otherwise provide a desired pharmacologic and/or physiologic effect. The precise dosage will vary according to a variety of factors such as subject-dependent variables (e.g., age, immune system health, etc.), the disease, and the treatment being effected.

For the disclosed probiotic compositions, as further studies are conducted, information will emerge regarding appropriate dosage levels for treatment of various conditions in various patients, and the ordinary skilled worker, considering the therapeutic context, age, and general health of the recipient, will be able to ascertain proper dosing. The selected dosage depends upon the desired therapeutic effect, on the route of administration, and on the duration of the treatment desired. For the disclosed probiotic compositions, the bacterial concentrations also depend on the type of bacterium. For the disclosed probiotic compositions, generally dosage levels of 10⁸-10¹² CFU daily are administered to mammals.

1. Formulations for Oral Administration

In some embodiments the compositions are formulated for oral delivery. Oral solid dosage forms are described generally in Remington's Pharmaceutical Sciences, 18th Ed. 1990 (Mack Publishing Co. Easton, Pa. 18042) at Chapter 89. Solid dosage forms include tablets, capsules, pills, troches or lozenges, cachets, pellets, powders, or granules or incorporation of the material into particulate preparations of polymeric compounds such as polylactic acid, polyglycolic acid, etc. or into liposomes. Such compositions may influence the physical state, stability, rate of in vivo release, and rate of in vivo clearance of the disclosed. See, e.g., Remington's Pharmaceutical Sciences, 18th Ed. (1990, Mack Publishing Co., Easton, Pa. 18042) pages 1435-1712 which are herein incorporated by reference. The compositions may be prepared in liquid form, or may be in dried powder (e.g., lyophilized) form. Liposomal or proteinoid encapsulation may be used to formulate the compositions. Liposomal encapsulation may be used and the liposomes may be derivatized with various polymers (e.g., U.S. Pat. No. 5,013,556). See also Marshall, K. In: Modern Pharmaceutics Edited by G. S. Banker and C. T. Rhodes Chapter 10, 1979. In general, the formulation will include the probiotic composition and inert ingredients which protect peptide in the stomach environment, and release of the biologically active material in the intestine.

Another embodiment provides liquid dosage forms for oral administration, including pharmaceutically acceptable emulsions, solutions, suspensions, and syrups, which may contain other components including inert diluents; adjuvants such as wetting agents, emulsifying and suspending agents; and sweetening, flavoring, and perfuming agents.

Controlled release oral formulations may be desirable. The agent can be incorporated into an inert matrix which permits release by either diffusion or leaching mechanisms, e.g., gums. Slowly degenerating matrices may also be incorporated into the formulation. Another form of a controlled release is based on the Oros therapeutic system (Alza Corp.), i.e., the drug is enclosed in a semipermeable membrane which allows water to enter and push drug out through a single small opening due to osmotic effects.

For a probiotic to successfully exert its benefit on the host's gut microbiota it should be able to remain viable during storage and also be capable of surviving, and potentially colonizing, the host's intestinal environment. Therefore, the probiotic composition should contain a concentration of live bacteria that is effective in causing benefits in the subject. Additionally, the capsule, pill, tablet, or syrup for oral administration should be stored in a manner so as to preserve its efficacy. Methods of storage include but are not limited to refrigeration, freezing, or storing at room temperature. If stored at room temperature, the probiotic should be stored in an air tight container.

2. Targeted Delivery

For enteral formulations, the location of release may be the small intestine (the duodenum, the jejunum, or the ileum), or the large intestine. The contents will be delivered via a method that ensures proper transit and survival to the targeted portion of the GI tract, depending on the targeted disease or disorder. For example, any products designed to treat or prevent Clostridium difficile infection will be formulated in a colon-targeted capsule, such as DRcaps™ from Capsugel®. Probiotics exert their main effect in the intestinal tract so in some embodiments, the release will avoid the deleterious effects of the stomach environment, either by protection of the agent (or derivative) or by release of the agent (or derivative) beyond the stomach environment. To ensure full gastric resistance, a coating impermeable to at least pH 5.0 is essential. Examples of common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D™, Aquateric™, cellulose acetate phthalate (CAP), Eudragit L™, Eudragit S™, and Shellac™. These coatings may be used as mixed films.

a. Delayed Release Formulation

Delayed release formulations can be created by coating a solid dosage form with a polymer film, which is insoluble in the acidic environment of the stomach, and soluble in the neutral environment of the small intestine.

The delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit® (Rohm Pharma; Westerstadt, Germany), including Eudragit® L30D-55 and L100-55 (soluble at pH 5.5 and above), Eudragit® L-100 (soluble at pH 6.0 and above), Eudragit® S (soluble at pH 7.0 and above, as a result of a higher degree of esterification), and Eudragits® NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

i. Time Controlled Capsules

In one embodiment, the probiotic compositions are formulated into time controlled capsules. The capsules may be manufactured using erodible capsule coating or specialized internal fillings so that the contents are released following transit of the capsule from the stomach to the upper or lower intestines. Time controlled release systems utilize measurements of gastric emptying and intestinal motility to ensure that the active ingredient stays protected until the capsule has cleared the stomach.

ii. pH Controlled Capsules

In one embodiment, the probiotic compositions are formulated into pH controlled capsules. The pH in the stomach ranges from 1 to 2 during fasting but increases after eating. In the small intestine, the pH is around 6.5 in the proximal regions and increases to about 7.5 in the distal portions. From there, the pH declines significantly as intestinal contents reach the cecum and then the colon, pH 6.4 and 5.7 respectively. These pH differences between portions of the GI tract can be utilized for the formulation of encapsulations based on polymers that are insoluble in low pH environments (e.g., the stomach) and soluble in higher pH environments (e.g. the lower digestive tract) (Philip & Philip, Oman Medical Journal, 25:79-87 (2003)).

iii. Enzyme Controlled Capsules

In some embodiments, the probiotic compositions are formulated into enzyme controlled capsules. Microbes that are specific to different portions of the GI tract are constantly fermenting the contents of their environment to generate the energy needed for their survival. Byproducts of the fermentation process include enzymes that can be specific to the microbe and the substance they are fermenting. As a result, capsules that will degrade only in the presence of site-specific enzymes can be designed (Philip & Philip, Oman Medical Journal, 25:79-87 (2003)).

III. Methods of Use

The disclosed probiotic compositions can be used, for example, to treat or prevent gastrointestinal diseases such as recurrent C. difficile infection, IBS, IBD, and antibiotic-associated microbial dysbiosis, to treat or prevent inflammation or an inflammatory response, to regulate intestinal homeostasis, or to regulate fluid and electrolyte balance in the intestines.

In some embodiments, the effect of the composition on a subject is compared to a control. For example, the effect of the composition on a particular symptom, pharmacologic, or physiologic indicator can be compared to an untreated subject or the condition of the subject prior to treatment. In some embodiments, the symptom, pharmacologic, or physiologic indicator is measured in a subject prior to treatment, and again one or more times after treatment is initiated. In some embodiments, the control is a reference level, or an average determined from measuring the symptom, pharmacologic, or physiologic indicator in one or more subjects that do not have the disease or condition to be treated (for example, healthy subjects). In some embodiments, the effect of the treatment is compared to a conventional treatment that is known in the art. For example, if the disease to be treated is cancer, the conventional treatment could be a chemotherapeutic agent.

A. Methods of Reducing Inflammation

Methods of using the disclosed compositions to treat or prevent inflammation in a subject in need thereof are provided. Methods typically include administering an effective amount of a butyric acid producing probiotic composition to a subject in need thereof.

One embodiment provides methods of treating an inflammatory response in a subject in need thereof. For example, the disclosed methods can be used to prophylactically or therapeutically inhibit, reduce, alleviate, or permanently reverse inflammation or an inflammatory response. In some embodiments, the disclosed compositions are effective in treating chronic inflammation or chronic inflammatory conditions. The term “chronic inflammation” as used herein refers to constantly recurring inflammation or inflammation that lasts for more than three months. An inflammatory response can be inhibited or reduced in a subject by administering to the subject an effective amount of the disclosed compositions.

In some embodiments, the disclosed probiotic compositions may be used to treat or prevent inflammatory conditions associated with gut microbial dysbiosis. It is believed that the anti-inflammatory effects of the disclosed compositions are likely mediated by increased levels of butyric acid in the intestinal lumen. Butyrate is known to inhibit the activation of NF-KB, which leads to a reduction in proinflammatory cytokines. It has also been shown that commensal gut microbiota may be associated with regulation of the immune system through their influence in the differentiation of lymphoid tissue, differentiation of different types of T cells and production of cytokines (Balzola et al., 2010; Ivanov et al., Cell, 139:485-498 (2009); Geuking et al., Immunity, 34:794-806 (2011)); Atarashi et al., Science, 331:337-341 (2011); Chung et al., Cell, 148:1578-1593 (2012)). Therefore, replacing commensal microbes that have been depleted in various gastrointestinal diseases could regulate the immune system. Further evidence indicates that butyrate produced by commensal bacteria plays a key role in their ability to modulate regulatory T cell (Tregs) differentiation and diversification (Furusawa et al., Nature, 446-450 (2013); Smith et al., Science, 341:569-573 (2013)).

Representative inflammatory conditions that can be inhibited or treated by the disclosed compositions include, but are not limited to, irritable bowel syndrome- constipation predominant, irritable bowel syndrome- diarrhea predominant, irritable bowel syndrome-mixed symptom, post-infectious irritable bowel syndrome, inflammatory bowel disease, including Crohn's disease and ulcerative colitis, antibiotic-induced diarrhea, C. difficile infection, and Celiac disease.

B. Methods of Regulating Intestinal Homeostasis

In one embodiment, the disclosed probiotic compositions can be used to regulate cellular homeostasis in the intestinal epithelium. The gastrointestinal tract is one of the largest interfaces between the host, environmental factors, and antigens passing through the body. The intestinal epithelium is exposed to all of the food, bacteria, and other material that pass through the intestinal tract on a daily basis. In order to combat the constant damage that could occur, the intestinal epithelium is renewed every 3-5 days. The balance between proliferation, differentiation, and terminal death of intestinal epithelial cells has been termed intestinal homeostasis. The disruption of these homeostatic processes is associated with the pathogenesis of various gastrointestinal diseases and disorders. Butyric acid has been implicated in the regulation of cellular proliferation and differentiation in the intestinal epithelium. In one embodiment, the disclosed butyrogenic probiotic compositions can regulate intestinal epithelial homeostasis.

C. Methods of Regulating Intestinal Fluid and Electrolyte Balance

Methods of using the disclosed probiotic compositions to treat or prevent fluid and electrolyte imbalance in a subject are provided. For example, in some embodiments, the disclosed compositions can be used to treat diarrhea.

The intestines are responsible for a bulk of the fluid reabsorption in the body. The absorption of water and solutes are tightly coupled, in other words, absorption of water is dependent on absorption of solutes, particularly sodium. Water diffuses in response to osmotic gradients established by sodium and enters into the intercellular space between cells, and eventually into capillaries in the villi. Disruptions in fluid and electrolyte balance in the intestines can lead to various gastrointestinal symptoms, including constipation and diarrhea.

Butyrate has known roles in the regulation of fluid and electrolyte uptake in the intestines. It has strong pro-absorptive, anti-secretory functions including stimulation of NaCl absorption by the action of two coupled transport systems on the intestinal brush border: Cl⁻/HCO₃⁻ and Na⁺/H⁺ and Cl⁻/butyrate and Na⁺/H⁺; and inhibition of Cl− secretion by blocking the activity of the co-transporter Na—K—2Cl (NKCCl) on the enterocyte basolateral membrane. In one embodiment, the disclosed butyric acid producing probiotic compositions can be used to regulate fluid and electrolyte uptake in the intestines. In another embodiment, the disclosed butyric acid producing compositions can be used in the treatment of conditions caused by intestinal fluid and electrolyte imbalance.

D. Diseases to be Treated

1. Inflammatory Bowel Disease

Methods of using the disclosed probiotic compositions to treat inflammatory bowel diseases are provided. Methods typically include administering an effective amount of a butyric acid producing probiotic composition to a subject in need thereof.

Several pieces of evidence suggest that alterations in the human gut microbiome play a major role in the pathogenesis of IBD. Studies have shown a reduction in Firmicutes and an increase in Proteobacteria in patients with IBD (Matsuoka & Kanai, Semin Immunopathol, 37:47-55 (2015)). There is a clear reduction in the biodiversity of bacteria, suggesting bacterial dysbiosis in this group of patients. However, it is unclear if dysbiosis is the cause or consequence of mucosal inflammation in the GI tract. Adults with IBD have also been shown to have a reduction in fecal or stool butyric acid and acetate (Huda-Faujan et al., Open Biochem J, 4:53-58 (2010)). Furthermore it has been shown that certain Clostridium species induce CD4 T cells in the regulation of the immune system in the GI tract. All these observations suggest a possible role for butyric acid producing probiotics in IBD.

In one embodiment, the subject in need thereof has IBD. In another embodiment, the subject in need thereof has Crohn's disease. In yet another embodiment, the subject in need thereof has ulcerative colitis.

2. Irritable Bowel Syndrome

Methods of using the disclosed probiotic compositions to treat irritable bowel syndrome are provided. Methods typically include administering an effective amount of a butyric acid producing probiotic composition to a subject in need thereof.

Recent evidence suggests a major role of dysbiosis in the human gut microbiome in the development of IBS. IBS is a complex disorder associated with changes in gut motility and the neuro-hormonal component of the enteric system. Reduction in Firmicutes and increase in Bacteroides has been documented in these patients (Kennedy et al., World J Gastroenterol, 12:1071-1077 (2014); Codling et al., Dig Dis Sci, 55:392-397 (2010)). A reduction in methane-producing bacteria and butyric acid production has been demonstrated in fecal samples collected from patients with IBS. Low grade inflammation in the colon has also been suspected in this group of patients in addition to bacterial dysbiosis. Butyric acid producing probiotics could be therapeutic for IBS due to the role of butyrate in regulating fluid and electrolyte uptake, proliferation and differentiation of epithelial cells, and anti-inflammation. Based on these beneficial functions of butyrate, probiotics incorporating butyrate-producing bacteria should be able to ameliorate symptoms of bloating and bowel alterations in IBS.

In some embodiments, the butyrogenic probiotics are used to treat subjects with IBS. In another embodiment, the subject in need thereof has mixed-symptom IBS, constipation-predominant IBS, or diarrhea-predominant IBS.

3. Antibiotic-Associated Diarrhea

Methods of using the disclosed probiotic compositions to treat antibiotic-associated diarrhea are provided. Methods typically include administering an effective amount of a butyric acid producing probiotic composition to a subject in need thereof.

The use of broad-spectrum (BS) antibiotics can have profound effects on the host microbiome. BS antibiotics are effective in killing target microbes, but also tend to induce collateral damage, in which crucial members of the host microbiota are depleted or eliminated. As previously stated, alterations in the microbiome are characterized by a reduction in biodiversity, reduction in Bifidobacteria and increase in Proteobacteria. These alterations in the human microbiome can last for months to years. These alterations have the potential to change metabolism and immunity in human subjects, including increasing an individual's susceptibility to infection by opportunistic commensals like Clostridial species (Johanesen et al., Genes, 6:1347-1360 (2015)). In one embodiment, administration of butyrogenic probiotics can ameliorate multiple metabolic and immunological consequences of long term antibiotic therapy.

In one embodiment, the subject in need thereof has antibiotic-associated diarrhea.

I claim:

1. A probiotic composition for the treatment of gastrointestinal dysbiosis comprising an effective amount of viable, non-pathogenic human gut microbes, wherein at least one of the microbes produces butyric acid.

2. The probiotic composition of claim 1 comprising:
an effective amount of at least one butyrogenic bacteria,
an effective amount of at least one Bifidobacterium spp.,
an effective amount of at least one Lactobacillus spp., and
an effective amount of S. boulardii.

3. The probiotic composition of claim 1 comprising 25% Butyrogenic bacteria, 25% Bifidobacterium spp, 25% Lactobacillus spp., and 25% S. boulardii.

4. The probiotic composition of claim 1 wherein the Butyrogenic bacteria is selected from the group containing Clostridium butyricum, Eubacterium limosum, Eubacterium rectale, Faecalibacterium prausnitzii, Roseburia faecis, and Roseburia intestinalis.

5. The probiotic composition of claim 1 comprising Clostridium butyricum, Faecalibacterium prausnitzii, Bifidobacterium spp., Lactobacillus spp., and S. boulardii.

6. The probiotic composition of claim 1 comprising Eubacterium limosum, Eubacterium rectale, Clostridium butyricum, Faecalibacterium prausnitzii, Bifidobacterium spp., Lactobacillus spp., and S. boulardii.

7. The probiotic composition of claim 1 comprising Roseburia faecis, Roseburia intestinalis, Clostridium butyricum, Faecalibacterium prausnitzii, Bifidobacterium spp., Lactobacillus spp., and S. boulardii.

8. The probiotic composition of claim 1 comprising Clostridium butyricum, Faecalibacterium prausnitzii, Bifidobacterium spp., Lactobacillus spp., and S. boulardii.

9. The probiotic composition of claim 1 further comprising a prebiotic.

10. The probiotic composition of claim 9 wherein the prebiotic is selected from the group containing inulin, arabinoxylan, xylose, soluble fiber dextran, soluble corn fiber, polydextrose, lactose, N-acetyl-lactosamine, glucose, galactose, fructose, rhamnose, mannose, uronic acids, 3′-fucosyllactose, 3′-sialylactose, 6′-sialyllactose, lacto-N-neotetraose, 2′-2′-fucosyllactose, trans-galactooligosaccharides, glucooligosaccharides, isomaltooligosaccharides, lactosucrose, polydextrose, pectin, soybean oligosaccharides, and arabinose, cellobiose, fructose, fucose, galactose, glucose, lactose, lactulose, maltose, mannose, ribose, sucrose, trehalose, xylobiose, xylooligosaccharide, D-xylose, and xylitol.

11. The probiotic composition of claim 9, wherein the prebiotic is dietary fiber.

12. The probiotic composition of claim 1, wherein the composition is formulated for oral administration.

13. The probiotic composition of claim 12, wherein the composition is formulated as a time controlled capsule.

14. The probiotic composition of claim 12, wherein the composition is formulated as a pH controlled capsule.

15. The probiotic composition of claim 12, wherein the composition is formulated as an enzyme controlled capsule.

16. The probiotic composition of claim 1, wherein the composition is formulated for rectal administration.

17. A method of treating gastrointestinal dysbiosis comprising administering to the subject in need thereof an effective amount of the probiotic composition of claim 1.

18. A method of treating diseases caused by gastrointestinal dysbiosis comprising administering to the subject in need thereof an effective amount of the probiotic composition of claim 1.

19. The method of claim 18, wherein the subject in need thereof has C. difficile infection, inflammatory bowel disease, Crohn's disease, ulcerative colitis, irritable bowel syndrome, or antibiotic-associated bacterial dysbiosis.

20. The method of claim 17 wherein the probiotic composition is administered orally.

21. The method of claim 17 wherein the probiotic composition is administered rectally.
